(19) United States
(12) Patent Application Publication
(10) Pub. No.: US 2022/0152351 A1
(43) Pub. Date: May 19, 2022

(54) CATHETER

(71) Applicant: TERUMO KABUSHIKI KAISHA, Tokyo (JP)

(72) Inventors: Kouta HAMURO, Fujinomiya-shi (JP); Tetsuya FUKUOKA, Ashigarakami-gun (JP)

(73) Assignee: TERUMO KABUSHIKI KAISHA, Tokyo (JP)

(21) Appl. No.: 17/587,180

(22) Filed: Jan. 28, 2022

Related U.S. Application Data
(63) Continuation of application No. PCT/JP2020/024079, filed on Jun. 19, 2020.

(30) Foreign Application Priority Data
Jul. 31, 2019 (JP) 2019-140688

(51) Int. Cl. A61M 25/01; A61M 25/00

(52) U.S. Cl. CPC A61M 25/0108; A61M 25/005

(57) ABSTRACT

Provided is a catheter that is excellent in visibility of a braid distal end portion inside and outside the body, is excellent in stenosed site crossability and trackability to a tortuous blood vessel, and prevents the braid from spreading out due to the stenosed site penetration. The catheter includes a shaft, the shaft includes an inner layer, an outer layer, and a braid that is disposed between the inner layer and the outer layer and includes a plurality of metal wires being woven together, the braid includes a welding portion at a further proximal side than a distal end of the shaft and in the vicinity of a distal end of the braid, and the shaft includes an radiopaque marker at a position separated from the welding portion, thereby preventing the braid from spreading out due to buckling of the shaft, and improving the blood vessel trackability.

### CROSS-REFERENCES TO RELATED APPLICATIONS

This application is a continuation of International Patent Application No. PCT/JP2020/024079 filed on Jun. 19, 2020, which claims priority to Japanese Patent Application No. 2019-1406881 filed on Jul. 31, 2019, the entire content of both of which is incorporated herein by reference.

### FIELD OF THE INVENTION

The present invention generally relates to a catheter.

### BACKGROUND DISCUSSION

A braid in which metal wires are woven together is typically embedded into a catheter for kink prevention, and a braid end portion in the vicinity of a distal end of the catheter is fixed with resin of an outer layer or an inner layer or a distal tip so as to prevent damage of a vascular wall.

In recent years, in some catheters, intersection points at the braid end portion are welded by a laser, thereby fixing the braid end portion.

### SUMMARY

U.S. Patent Application Publication No. 2008/0125752 discloses a catheter in which a braid end portion is welded. Moreover, Japanese Patent Application Publication No. 2014-144163 discloses a catheter in which a distal tip is provided at a distal end of a shaft, and in order to prevent the distal tip from pulling out from the shaft and falling off, a bulging portion larger than the thickness of the metal wire is formed in the braid end portion.

However, in this method, an interface of the resin is present between the distal tip and the shaft, and the thickness of the outer layer of the bulging portion is reduced, so that the tensile strength may decreased.

Moreover, even the distal tip using the resin containing an X-ray contrast agent becomes thin-walled when the catheter is thin, so that it is difficult to visually recognize the distal tip under the radiography.

The distal end position of the braid cannot be recognized in such a state, so that the distal tip of the catheter may be brought into contact with a vascular wall to cause separation of the vascular wall.

(1) A catheter according to one aspect comprises a shaft including a distal end and a proximal end, in which the shaft includes an inner layer, an outer layer, and a braid that is disposed between the inner layer and the outer layer and includes a plurality of metal wires that are woven together, the braid includes a welding portion at a further proximal side than the distal end of the shaft that welds intersection points of the metal wires in a vicinity of a distal end of the braid, and the shaft includes a radiopaque marker at a position separated from the welding portion to the proximal side.

(2) In the catheter according to the above (1), the radiopaque marker may have a length in a long axis direction of the shaft, and the length in the long axis direction of the radiopaque marker may be longer than a distance from the welding portion to a distal end of the radiopaque marker.

(3) In the catheter according to the above (1) or (2), the radiopaque marker may be a coil wire, and a diameter of the coil wire may be less than a distance from a distal end of the welding portion to the distal end of the radiopaque marker.

With the catheter disclosed here, the welding portion of the intersection points that is located at the distal side of the braid is located at the further distal side than the distal end of the radiopaque marker, so that the position of the welding portion can be visually observed outside the body, and the radiopaque marker of the catheter inside the body can be visually recognized under the radiography. Therefore, the catheter is easily inspected when being manufactured, and can be accurately positioned when being moved forward in the unit of millimeters during surgery.

In addition, a distal end of the radiopaque marker is separated from the welding portion, so that the distal end portion of the catheter becomes more flexible to improve the trackability and the branch selectivity in a complicated and thin blood vessel such as a hepatic artery.

Accordingly, the catheter easily reaches a lesion area to shorten the surgery time, thereby allowing the reduction in the burden of a patient, and the reduction in labor costs.

According to another aspect, a catheter comprises a tubular shaft including a distal end and a proximal end at opposite axial ends of the tubular shaft, with the tubular shaft possessing an inner surface facing a hollow interior of the tubular shaft and an outer surface. The tubular shaft includes an inner layer, an outer layer, and a braid positioned radially inwardly of the outer surface of the tubular shaft and radially outwardly of the inner surface of the tubular shaft, with the braid including a plurality of metal wires that are woven together. The braid includes a distal end portion terminating at a distal-most end of the distal portion of the braid, wherein the plurality of metal wires in the distal portion of the braid are welded together and constitute a welding portion, with the distal end of the tubular shaft being positioned distal of a distal-most end of the welding portion of the braid. The shaft includes a radiopaque marker positioned radially inwardly of the outer surface of the tubular shaft and radially outwardly of an outer surface of the braid, with the radiopaque marker including a distal-most end and a proximal-most end, and a proximal-most end of the welding portion being positioned distal of the distal-most end of the radiopaque marker, and the braid extending proximally beyond a proximal-most end of the radiopaque marker.

### BRIEF DESCRIPTION OF THE DRAWINGS

FIG. 1 is a cross-sectional view of a distal end portion of a catheter according to a first embodiment of the invention.

FIG. 2 is a cross-sectional view of a distal end portion of a catheter according to a second embodiment of the invention.

### DETAILED DESCRIPTION

Set forth below with reference to the accompanying drawing figures is a detailed description of embodiments of a catheter representing examples of the inventive catheter disclosed here. The dimensions or scales on the drawings may be exaggerated or different from actuality/reality for convenience of description and illustration. In the description that follows, a side or end of a catheter to be inserted into a living body lumen is referred to as a “distal side” or “distal end”, and a side at which the catheter is operated is referred to as a “proximal side” or “proximal end”.

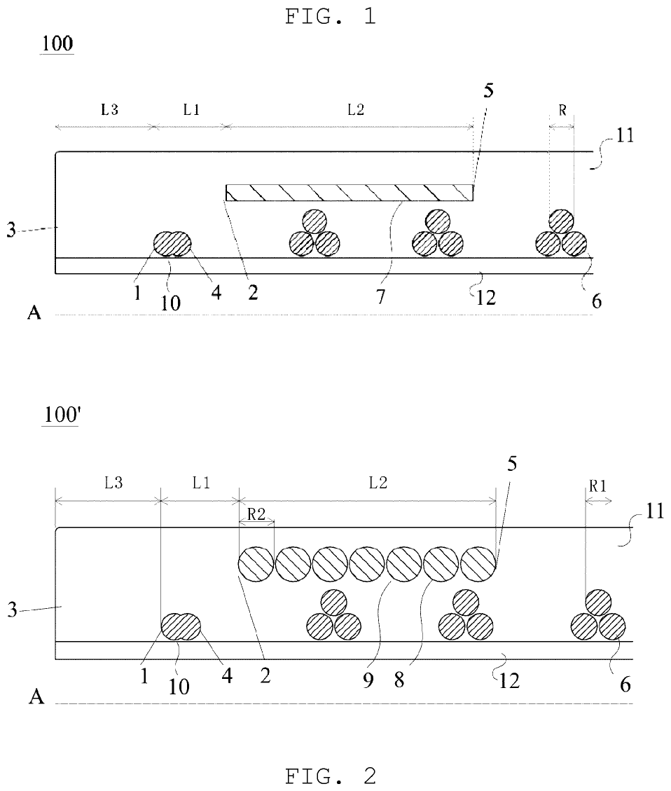

A shaft 100 of a catheter as a first embodiment illustrated in FIG. 1 includes an outer layer 11 and an inner layer 12, and includes a braid 6 therebetween in which metal wires and the like are woven together. As shown in FIG. 1, the shaft 100 is a tubular shaft in which the interior surface of the shaft 100 surrounds a hollow space.

A plurality of metal wires are woven together to form the braid 6 in a tubular shape so as to have gaps penetrating between an inner peripheral surface or inner layer of the braid and an outer peripheral surface or outer layer of the braid. The braid 6 includes a welding portion or welded portion 10 formed in such a manner that intersection points of the metal wires mutually crossing one another are welded together.

This helps prevent the braid 6 from spreading out due to the shaft 100 becoming buckled when a shaft distal end 3 is brought into contact with a hard stenosed site or the like, and a part of the metal wires from breaking through the outer layer 11 of the shaft 100.

The welding portion 10 is preferably positioned on a distal end of the braid 6, but may be welded on a position at a further proximal side than the distal end of the braid and at a further distal side than a distal end of an radiopaque marker, so as to be more firmly fixed.

Alternatively, in order to reduce a level difference in physical properties between the welding portion 10 and the shaft distal end 3, the metal wires may protrude from the welding portion 10 to the distal side (i.e., the metal wires may extend distally beyond the welding portion 10).

The welding portion 10 includes a welding portion distal end 1 and a welding portion proximal end 4. In the present embodiment, the braid 6, one of which is a single wire and the other of which is a multiple wire including two metal wires, is formed in such a manner that the single wire and the multiple wire are crossed and woven together. Note that, single wires or multiple wires may be woven together to form the braid 6. Moreover, metal wires having different thicknesses and types may be woven together to form the braid 6.

Examples of materials for the braid 6 may include tungsten or stainless steel wire. The metal wire forming the braid 6 may have any desired thickness, and preferably has an outer diameter R of 5 to 100 μm, preferably 15 to 60 μm. The cross-section of the metal wire in the braid 6 is circular, but may also be elliptical, rectangular, or oval.

In the first embodiment illustrated in FIG. 1, the welding portion distal end 1 is disposed proximal of the shaft distal end 3 by a distance L3. In addition, a distal end 2 of a tubular marker 7 serving as a radiopaque marker is disposed at a position that is proximal of the welding portion distal end 1 by a distance L1. The distance L1 is less than a distance between the distal end 2 and a proximal end 5 of the tubular marker 7, in other words, a length L2 in a longitudinal axis direction of the tubular marker 7. The longitudinal axis direction is a direction along the longitudinal axis extending between a distal end and a proximal end of the shaft 100, for example, a direction along a central axis A of a lumen of the shaft 100.

Therefore, even when the shaft distal end 3 is brought into contact with a stenosed site, the braid distal end does not spread out between the welding portion distal end 1 and the distal end 2 of the tubular marker 7 by the force transmitted from the shaft distal end 3, and the force that is transmitted from the shaft distal end 3 is transmitted to the tubular marker 7 with high efficiency. Therefore, the shaft 100 can pass through the stenosed site without buckling.

The tubular marker 7 (radiopaque marker) is disposed at a position separated or spaced from the welding portion 10 toward the proximal side. That is, the distal end of the tubular marker 7 is positioned proximal of the welding portion 10. Therefore, a position of the welding portion 10 can be visually observed through a transparent material outside the body, and the tubular marker 7 of the catheter inside the body can also be visually recognized under the radiography. Therefore, the catheter is easily inspected during manufacturing, and can be accurately positioned when being moved forward in small units such as the unit of millimeters during surgery. In addition, a distal end of the tubular marker 7 is separated or spaced from the welding portion 10, so that the distal end portion of the catheter becomes more flexible to improve the trackability and the branch selectivity in a complicated and thin blood vessel such as a hepatic artery. Accordingly, the catheter easily reaches a lesion area to shorten the surgery time, thereby allowing a reduction in the burden on a patient, and a reduction in labor costs. FIG. 1 also shows that the tubular marker 7 includes a proximal-most end that is a free end of the tubular marker 7, and this proximal-most end of the tubular marker 7 is distal of the proximal end portion of the braid.

The tubular marker 7 having a cylindrical shape is formed by using an iridium alloy and the like. The thickness of the tubular marker 7 is 5 to 100 μm, preferably 15 to 60 μm. The length in the longitudinal axis direction (axial direction) of the tubular marker 7 is 0.5 to 10 mm, preferably 1 to 3 mm. The tubular marker 7 may be fitted into the shaft 100 or embedded into the shaft 100 with a resin.

A shaft 100′ according to a second embodiment illustrated in FIG. 2 includes a coil marker 9 as the radiopaque marker in which a coil wire 8 made of metal that is recognizable under radiography is wound, and is common to that in the first embodiment other than the coil marker 9, so that an explanation thereof is omitted.

A distal end 2 of the coil marker 9 is disposed at a position distant by a distance L1 from the welding portion distal end 1 to the proximal side, and the distance L1 is less than a length L2 in the longitudinal axis (axial) direction of the coil wire 8. That is, the distal end 2 of the coil marker 9 is positioned proximal of the distal end 1 of the welding portion 10 by an axial distance L1, and this axial distance L1 is less than the axial length L2 of the coil wire 8.

The coil marker 9 is flexible and easily deforms, so that a distal end portion of the shaft 100′ has improved trackability to the bent blood vessel.

The coil marker 9 is formed by using a gold alloy and the like. An outer diameter R2 of the wire or the coil wire 8 that is wound or coiled to form the coil marker 9 is 5 to 100 μm, preferably 15 to 60 μm. The coil wire 8 is coiled around the shaft 100′. That is, the coil wire 8 is a wire coiled or wound about the longitudinal axis of the shaft 100′ to form a tubular-configuration or a tubular-shape. The coil wire 8 is wound such that a length L2 in the longitudinal axis direction of the coil marker 9 (i.e., the axial length) is 0.5 to 10 mm, preferably 1 to 3 mm. The outer diameter R2 of the coil wire 8 is not specially limited, but is preferably less than the distance L1 from the welding portion distal end 1 to the distal end 2 of the coil marker 9 (radiopaque marker). This prevents the coil wire 8 from becoming too thick, and the coil marker 9 is flexible and easily deforms, so that a distal end portion of the shaft 100′ has improved trackability to the bent blood vessel. FIG. 2 also shows the outer diameter R1 of the wire forming the braid 6.

Therefore, the shaft 100′ can pass through a bifurcated blood vessel of the coronary artery and a tortuous blood vessel or a branched blood vessel of the hepatic artery by following the guide wire.

The detailed description above describes embodiments of a catheter representing examples of the inventive catheter disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A catheter comprising:
a shaft including a distal end and a proximal end;
the shaft including an inner layer, an outer layer, and a braid that is disposed between the inner layer and the outer layer, the braid including a plurality of metal wires that are woven together;
the braid including a welding portion at which is welded intersection points of the metal wires in a vicinity of a distal end of the braid, the welding portion being located proximal of the distal end of the shaft; and
the shaft including a radiopaque marker at a position separated on a proximal side from the welding portion.

2. The catheter according to claim 1, wherein the radiopaque marker has a length in an axial direction of the shaft, and the length of the radiopaque marker in the axial direction being greater than a distance from the welding portion to a distal end of the radiopaque marker.

3. The catheter according to claim 2, wherein the radiopaque marker is a coil wire comprised of a wire that is wound in a coil, and an outer diameter of the wire being less than a distance from a distal end of the welding portion to the distal end of the radiopaque marker.

4. The catheter according to claim 1, wherein the radiopaque marker is a coil wire comprised of a wire that is wound in a coil, and an outer diameter of the wire being less than a distance from a distal end of the welding portion to the distal end of the radiopaque marker.

5. The catheter according to claim 1, wherein the radiopaque marker is a tube made of radiopaque material.

6. The catheter according to claim 1, wherein a distal-most end of the welding portion is positioned proximal of a distal-most end of the shaft.

7. The catheter according to claim 1, wherein the braid is positioned radially between an inner surface of the shaft and an inner surface of the radiopaque marker.

8. The catheter according to claim 1, wherein the radiopaque marker includes a proximal-most end that is a free end of the radiopaque marker, the proximal-most end of the radiopaque marker being positioned distally of a proximal end portion of the braid.

9. The catheter according to claim 1, wherein the radiopaque marker possesses an axial length of 0.5 mm to 10 mm.

10. The catheter according to claim 1, wherein the radiopaque marker is tubular in configuration and possesses a proximal-most end and a distal-most end, the radiopaque marker being configured and positioned so that: i) the distal-most end of the radiopaque marker is proximal of the distal end of the shaft; ii) the braid extends distally beyond the distal-most end of the radiopaque marker; and iii) the braid extends proximally beyond the proximal-most end of the radiopaque marker.

11. The catheter according to claim 1, wherein the tubular shaft includes a central axis, each of the metal wires forming the braid possessing an outside diameter, the radiopaque marker possessing a distal-most end and the welding portion possessing a distal-most end, the distal-most end of the welding portion being positioned distally beyond the distal-most end of the braid by an axial distance parallel to the central axis of the shaft, the outer diameter of the metal wires being less than the axial distance.

12. A catheter comprising:
a tubular shaft including a distal end and a proximal end at opposite axial ends of the tubular shaft, the tubular shaft possessing an inner surface facing a hollow interior of the tubular shaft and an outer surface;
the tubular shaft including an inner layer, an outer layer, and a braid positioned radially inwardly of the outer surface of the tubular shaft and radially outwardly of the inner surface of the tubular shaft, the braid including a plurality of metal wires that are woven together;
the braid including a distal end portion terminating at a distal-most end of the distal portion of the braid, the plurality of metal wires in the distal portion of the braid being welded together and constituting a welding portion, the distal end of the tubular shaft being positioned distal of a distal-most end of the welding portion of the braid; and
the shaft including a radiopaque marker positioned radially inwardly of the outer surface of the tubular shaft and radially outwardly of an outer surface of the braid, the radiopaque marker including a distal-most end and a proximal-most end, a proximal-most end of the welding portion being positioned distal of the distal-most end of the radiopaque marker, the braid extending proximally beyond a proximal-most end of the radiopaque marker.

13. The catheter according to claim 12, wherein the radiopaque marker has a length in an axial direction of the shaft, and the length of the radiopaque marker in the axial direction being greater than a distance from the proximal-most end of the welding portion to a distal end of the radiopaque marker.

14. The catheter according to claim 13, wherein the radiopaque marker is a coil wire comprised of a wire that is wound in a coil, an outer diameter of the wire constituting the radiopaque marker being less than a distance from a distal-most end of the welding portion to the distal-most end of the radiopaque marker.

15. The catheter according to claim 12, wherein the radiopaque marker is a coil wire comprised of a wire that is wound in a coil, an outer diameter of the wire constituting the radiopaque marker being less than a distance from a distal-most end of the welding portion to the distal-most end of the radiopaque marker.

16. The catheter according to claim 12, wherein the radiopaque marker is a tube made of radiopaque material.

17. The catheter according to claim 12, wherein the radiopaque marker possesses an axial length of 0.5 mm to 10 mm.

18. The catheter according to claim 12, wherein the tubular shaft includes a central axis, each of the metal wires forming the braid possessing an outside diameter, the distal-most end of the welding portion being positioned distally beyond the distal-most end of the braid by an axial distance parallel to the central axis of the shaft, the outer diameter of the metal wires being less than the axial distance.
